(19) United States
(12) Patent Application Publication
(10) Pub. No.: US 2008/0221552 A1
(43) Pub. Date: Sep. 11, 2008

(54) AGENT DELIVERY PERFUSION CATHETER

(75) Inventor: Michael J. Leonard, San Luis Obispo, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: ABBOTT CARDIOVASCULAR SYSTEMS INC., Santa Clara, CA (US)

(21) Appl. No.: 11/684,058

(22) Filed: Mar. 9, 2007

(51) Int. Cl. A61M 25/10

(52) U.S. Cl. 604/509

(57) ABSTRACT

An agent delivery catheter and method, configured to deliver an agent to an inner surface of a patient's body lumen by forming an agent containment pocket along the inner surface of the body lumen wall, while minimizing ischemic conditions during the procedure.

### CROSS-REFERENCES TO RELATED APPLICATIONS

None

### BACKGROUND OF THE INVENTION

The present invention relates generally to medical devices, and more particularly to a catheter for delivery of an agent to the coronary or peripheral vasculature.

In the treatment of diseased vasculature, therapeutic agents have commonly been administered, typically as part of other interventional therapies such as angioplasty or stent delivery. Local, as opposed to systemic delivery is a preferred method of treatment in that smaller total levels of medication are administered in comparison to systemic dosages yet are concentrated at a specific site. As a result, local delivery produces fewer side effects and achieves more effective results.

A variety of methods and devices have been proposed for percutaneous drug delivery to a diseased region of the vasculature. For example, catheters having porous balloons can be used to deliver a therapeutic agent infused into the inflatable interior of the porous balloon and through the porous wall of the balloon. Alternatively, prostheses such as stents or other implantable devices provide for local drug delivery when coated or otherwise made to include a therapeutic agent which elutes from the implanted prosthesis. Another suggested method involves the use of one or more catheters having multiple balloons. The diseased region is isolated by inflating the balloons on either side of the diseased region, and the therapeutic agent is infused through a lumen of the catheter shaft and into the isolated diseased region from a delivery port on the catheter shaft located between the balloons. However, the balloons inflated against the vessel wall occlude the vessel, and thus create ischemic conditions there along and distal thereto.

In order to properly position the distal end of a drug delivery catheter in a patient's tortuous distal vasculature, the catheter should preferably have a low-profile, flexible distal section despite also having the necessary structural components required for the drug delivery at the operative distal end of the catheter. One difficulty has been providing for a large amount of drug taken-up and retained at the diseased site, while minimizing the wash out of significant amounts of drug downstream of the treatment site. Drug wash out reduces the efficiency of local intravascular drug delivery, in addition to causing potentially harmful systemic exposure to the drug. Therefore, it would be a significant advance to provide an improved device and method for providing therapy to a desired location within a patient's body lumen.

### SUMMARY OF THE INVENTION

The invention is directed to an agent delivery catheter and method configured to deliver an agent to an inner surface of a patient's body lumen by forming an agent containment pocket along the inner surface of the body lumen wall, while minimizing ischemic conditions during the procedure.

A catheter of the invention generally includes an elongated shaft having an inflation lumen, an agent delivery lumen which is in fluid communication with an agent delivery distal port, and a distal shaft section which has a proximal sealing member and a distal sealing member secured thereto, and a tubular membrane extending from the proximal sealing member to the distal sealing member. Each sealing member preferably comprises a ring which is biased to radially self-expand from a collapsed to a radially expanded configuration upon removal of a radially restraining force, and a doughnut-shaped balloon on the self-expanding ring which has an inflatable interior chamber in fluid communication with the inflation lumen. The tubular membrane has an outer surface, and an inner surface defining a perfusion channel extending through the membrane, with the shaft extending in the perfusion channel, and the agent delivery lumen extending across the membrane (e.g., to a port in the sidewall of the membrane) from the inner towards the outer surface thereof, so that the catheter is configured to deliver an agent to an agent containment pocket which extends along the inner surface of the patient's body lumen between the expanded sealing members and which is formed by the outer surface of the membrane and the expanded sealing members.

Each balloon preferably encircles and extends fully around (i.e., 360° around) the circumference of the shaft. As a result, when the balloons are inflated against an inner surface of the patient's vessel, the balloons exert a sealing force against the vessel wall which contains the agent infused from the agent lumen into the agent containment pocket extending along the catheter membrane. Although the agent containment pocket thus isolates the region of the patient's vessel from blood flow, the perfusion channel allows for blood to continue flowing in the patient's body lumen through the sealing members. Additionally, the balloons each preferably contact a relatively small length of vessel wall unlike conventional occlusion, dilation or stent delivery balloons with a longer working length section. Consequently, when the sealing members are deployed, only that relatively small length of vessel wall is separated by the balloon from both the blood flow of the body lumen and the agent flow of the agent delivery lumen. That is, the vessel wall along the containment pocket is exposed at least to agent, and the rest of the vessel wall remains exposed to the blood flow of the body lumen. Thus, the catheter minimizes ischemic conditions caused by its use in delivering agent to the vessel wall.

The catheter is preferably configured such that the resulting agent containment pocket extends fully around the inner circumference of the body lumen. Thus, with the sealing members expanded against the vessel wall, the catheter allows for delivering agent to the entire circumference and length of the vessel wall extending between the deployed sealing members. As a result, the catheter maximizes the amount of the vessel wall directly exposed to the agent, and, moreover, thus minimizes the duration required for the procedure (while nonetheless allowing for the procedure to be done over longer periods of time due to the non-occlusive nature of the catheter of the invention).

A method of the invention generally involves introducing within a patient's body lumen a catheter comprising an elongated shaft having an inflation lumen and an agent delivery lumen, a distal shaft section of the elongated shaft having a proximal sealing member and a distal sealing member secured thereto, and a tubular membrane extending from the proximal sealing member to the distal sealing member, having an outer surface, and an inner surface defining a perfusion channel therethrough, with the shaft extending in the perfusion channel. The agent delivery lumen extends across the membrane from the inner towards the outer surface thereof, typically to a single agent delivery port in a sidewall of the membrane, such that agent can be delivered along an outer surface of the membrane. The method includes radially expanding the sealing members against an inner surface of the body lumen wall, and delivering an agent to an agent containment pocket which extends along the inner surface of the patient's body lumen wall between the deployed sealing members and which is formed by the outer surface of the membrane and the deployed sealing members. Each sealing member typically comprises a radially self-expanding component and an inflatable component. Preferably, radially self-expanding each sealing member causes the tubular membrane to radially expand and open the perfusion channel therethrough, and then inflating each sealing member to complete the deployment of the sealing members exerts a sealing force against the body lumen wall. The inflatable components (e.g., doughnut-shaped balloons) of the proximal and distal sealing members typically inflate together. However, the catheter can alternatively be configured with multiple inflation lumens or valves to allow for independent inflation. Similarly, the balloons can be configured to inflate at different inflation pressures, with, for example, the proximal balloon inflating first at a lower inflation pressure than the distal balloon, to thus allow for purging/displacing at least some blood from the agent containment pocket.

A variety of suitable agents, including diagnostic and therapeutic agents, can be delivered to the agent containment pocket using the catheter and method of the invention. The agent is typically a therapeutic agent for restenosis. In a presently preferred embodiment, the agent is selected from the group consisting of an anti-inflammatory agent, a lipid transport aid agent such as ApoA1, and a drug for destroying macrophages.

A catheter of the invention allows for improved delivery of an agent to a patient's vessel wall by allowing for agent delivery to take place over a desired period of time while preventing or minimizing disadvantageous, damaging ischemia in the vessel wall. The catheter maximizes agent up-take in the vessel wall, while nonetheless minimizing wash out of the agent in the blood vessel. Moreover, the catheter is preferably highly maneuverable, to facilitate positioning the operative distal end at a desired location within the body lumen. These and other advantages of the invention will become more apparent from the following detailed description of the invention and accompanying exemplary drawings.

### BRIEF DESCRIPTION OF THE DRAWINGS

FIG. 1 is an elevational, partially in section, view of an agent delivery perfusion catheter embodying features of the invention, having sealing members in a collapsed configuration within a patient's body lumen.

FIG. 2 illustrates a perspective view of the catheter of FIG. 1 with the sealing members in an expanded configuration.

FIGS. 3-6 are transverse cross sections of FIG. 2, taken along lines 3-3, 4-4, 5-5, and 6-6, respectively.

FIG. 7 illustrates the catheter of FIG. 1, partially in section, with the catheter sealing members in a deployed configuration during delivery of an agent to the body lumen wall.

FIGS. 8 and 9 illustrate transverse cross sections of FIG. 7, taken along lines 8-8 and 9-9, respectively.

### DETAILED DESCRIPTION OF THE PREFERRED EMBODIMENTS

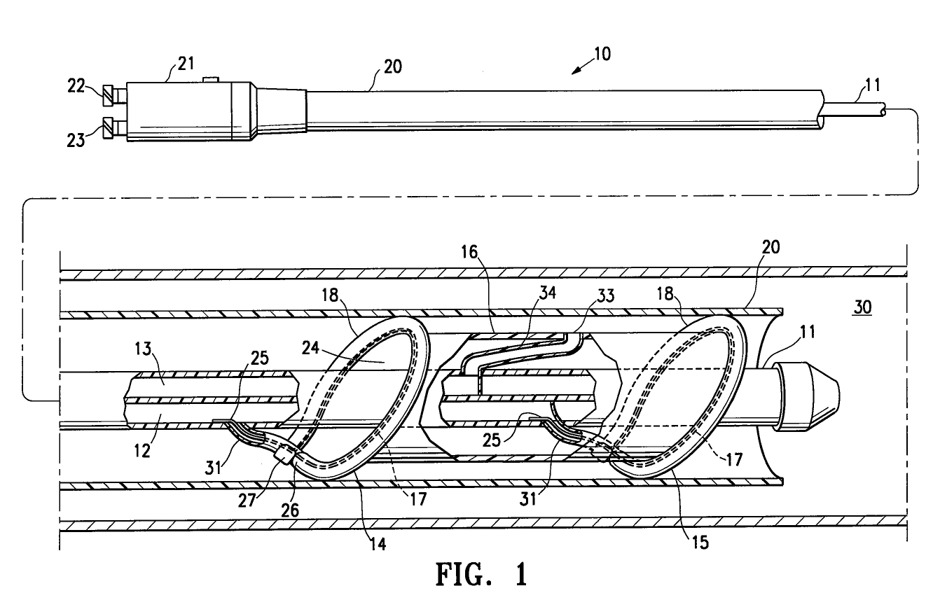

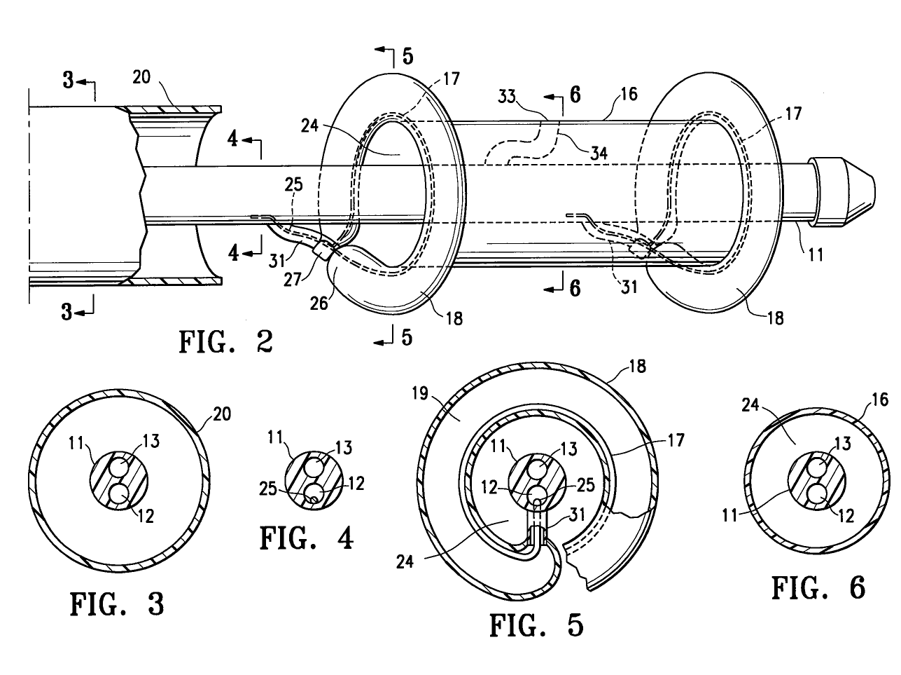

FIG. 1 illustrates an elevational, partially in section, view of an agent delivery perfusion catheter 10 embodying features of the invention, generally comprising an elongated shaft 11 having an inflation lumen 12 and an agent delivery lumen 13, a proximal sealing member 14, a distal sealing member 15, and a tubular membrane 16 extending from the proximal sealing member 14 to the distal sealing member 15. In the illustrated embodiment, each sealing member 14, 15 reversibly expands from a low profile configuration to a deployed configuration, and comprises a ring 17 which is biased to radially self-expand from a collapsed configuration to a radially expanded configuration upon removal of a radially restraining force therefrom, and a doughnut-shaped (toroidal) balloon 18 which is on the ring 17 and which has an inflatable interior chamber 19 (see FIG. 5) in fluid communication with the inflation lumen 12 of the catheter shaft 11. The rings 17 are shown as dash lines within the interior chamber 19 of the balloons 18 in FIG. 1. FIG. 1 illustrates the sealing members 14, 15 in a low profile collapsed configuration within a patient's body lumen 30, and FIG. 2 illustrates a perspective view of the catheter of FIG. 1 with the sealing members 14, 15 in the deployed configuration in which the rings 17 are radially expanded and the balloons 18 are inflated. FIGS. 3-6 illustrate transverse cross sections of FIG. 2, taken along lines 3-3, 4-4, 5-5 and 6-6, respectively.

In the illustrated embodiment, the radially restraining force is provided by a sheath member 20 which is disposed on the shaft 11 and which extends over and thereby collapses the self-expanding rings 17 in an advanced configuration, as illustrated in FIG. 1. The sheath member 20 and shaft 11 are slidably disposed relative to one another, and the sheath has a retracted configuration in which the distal end of the sheath is proximal to each ring 17 and each ring is thereby radially expanded to the radially expanded configuration, as illustrated in FIG. 2. The sheath member 20 typically has a tubular body extending to the proximal end of the shaft 11, and the proximal end of the sheath 20 can be manipulated outside of the patient's body lumen to advance or retract the distal end of the sheath 20 over the shaft 11. For example, a proximal adapter handle 21 on the proximal end of the shaft 11 is configured for longitudinally displacing the shaft 11 and sheath 20 relative to one another. Such handle mechanisms are generally known and typically include a thumb wheel, trigger, lever or other activation mechanism for advancing and/or retracting a shaft. A lock mechanism to releasably secure the sheath 20 to the elongated shaft 11 may be provided, typically as part of the proximal handle 21. A variety of suitable mechanisms may be used to releasably lock the sheath 20 to the elongated shaft 11 as are conventionally known, typically in the form of a clamp or other locking mechanism at or near the proximal end of the sheath 20.

Each ring 17 is located within the inflatable interior chamber 19 of its respective balloon 18. The ring is preferably dimensioned and positioned to hug the inner circumference of the interior chamber 19 of the inflated balloon 18 as best illustrated in FIG. 5. The ring 17 is preferably not bonded to the wall of the balloon 18, in order to minimize any disadvantageous affects of the ring on balloon integrity or expansion of the balloon 18 during inflation thereof. The ring 17 is typically secured to the shaft 11 by a tether member 25 extending there between. Specifically, in the illustrated embodiment, the tether member 25 and ring 17 are an integral, one-piece unit formed of a length of wire having a first section extending in the inflation lumen 12 to form the tether member 25 and a second, loop section forming the self-expanding ring 17 in the balloon interior chamber 19. The proximal end of the wire is fixedly secured to an inner surface of the shaft 11 within the inflation lumen 12, typically by an adhesive or fusion bonding to an outer polymeric layer on the wire, or wound to enable fixation of the wire. The self-expanding ring 17 (and tether 25) is typically formed of a nickel-titanium alloy wire, heat-set to form the ring with the desired radial dimension and biased to self-expand from the collapsed to the radially expanded configuration. The NiTi wire preferably has a round transverse shape, although a variety of suitable configurations could alternatively be used such as a flat ribbon transverse shape. The rings 17 can alternatively be formed of stainless steel, or cobalt-chrome-moly alloy, although shape memory or superelastic alloys such as NiTi are preferred due at least in part to the high acceptable strain limits.

Each balloon 18 is configured to connect to a source of inflation fluid. In the illustrated embodiment, a short conduit 31 extends at a angle radially away from the shaft 11 between each balloon 18 and the shaft 11, so that the inflatable interior chamber 19 of the balloon 18 is in fluid communication with the inflation lumen 12. Each doughnut-shaped balloon 18 is formed from a length of conventional balloon tubing which is formed into the doughnut shape typically by heat-forming in a curvilinear mold. The inner surface of a first end 26 of the balloon tubing is sealingly secured to the end of the conduit 31 so that the interior chamber of the balloon tubing is in fluid communication with the section of the inflation lumen 12 defined by the conduit 31. The outer surface of the opposite end 27 of the balloon tubing is wrapped partially on the conduit and sealingly secured thereto adjacent to the bonded first end of the balloon tubing as best illustrated in FIGS. 2 and 9, such that the balloon ends are directly adjacent to one another to form a 360°, doughnut-shaped balloon.

An inner surface of the tubular membrane 16 defines a perfusion channel 24 therethrough. Specifically, with the sheath 20 retracted such that the rings 17 are radially expanded in a patient's blood vessel, blood flow in the vessel is able to flow past the sealing members 14, 15 through the membrane 16. Inflation of the balloons 18 to fully deploy the sealing members 14, 15 may further open perfusion channel 24 within the membrane 16. The membrane collapses with the rings 17 to a collapsed configuration so that the membrane is positioned within the sheath 20 and the perfusion channel 24 is closed when the sheath 20 is in the advanced configuration. Although the sealing members 14, 15 and membrane 16 are illustrated in FIG. 1 radially spaced from the underlying section of the shaft 11 for ease of illustration, it should be understood that the sealing members 14, 15 and membrane 16 typically collapse down onto the shaft in the collapsed configuration.

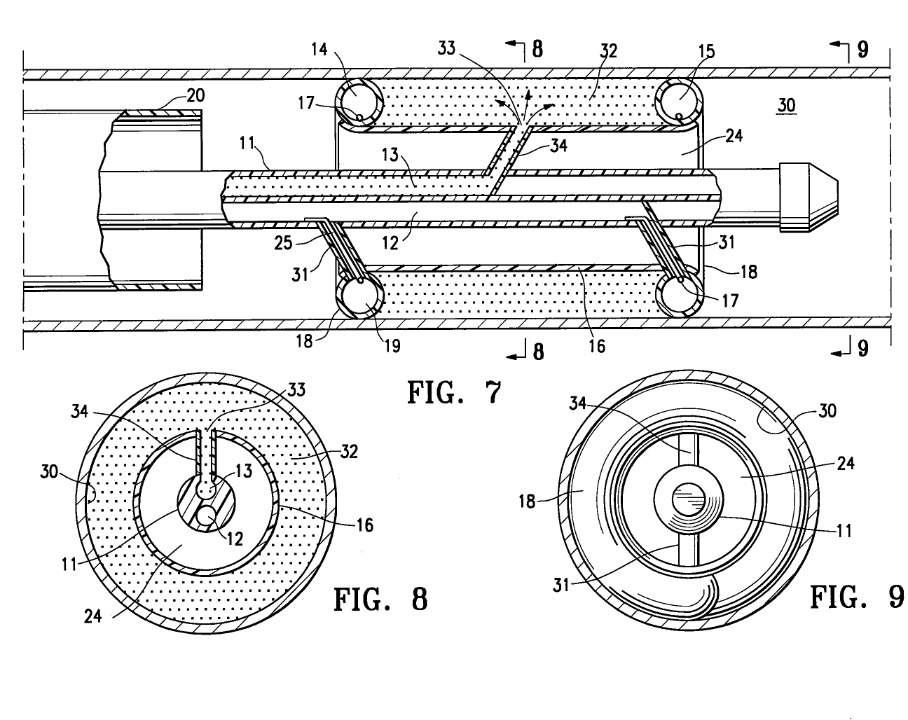

FIG. 7 illustrates the catheter 10 of FIG. 1, with the catheter sealing members 14, 15 in the deployed configuration, during delivery of an agent to the wall of the patient's body lumen 30. In FIG. 7, the membrane 16, rings 17, and balloons 18 are illustrated in longitudinal cross section, showing the shaft 11 extending within the perfusion channel 24. With the sealing members 14, 15 in the deployed configuration in the patient's body lumen 30, an agent containment pocket 32 is defined, which extends along the inner surface of the wall of the body lumen 30 between the deployed sealing members 14, 15. The agent containment pocket 32 is formed by the outer surface of the membrane 16 and the deployed sealing members 14, 15. Depending on how the membrane 16 joins to each sealing member 14, 15, the agent containment pocket 32 may be defined by the membrane 16, or alternatively by the membrane 16 and a portion of the sealing members 14, 15 as in the embodiment illustrated in FIG. 7 in which the outer surface of the membrane 16 is secured to the interior channel surface of the doughnut-shaped balloons 18 (i.e., the membrane extends into the hole of the doughnut-shaped balloon, and bonds to an outer surface of the balloon). The membrane in the embodiment illustrated in FIG. 7 has a length shorter than the distance from the proximal end of the proximal balloon to the distal end of the distal balloon. However, the membrane can have a longer or slightly shorter length than in the illustrated embodiment.

Preferably, the shaft 11 is coaxially centered within the expanded membrane 16, as best illustrated in FIG. 8 showing a transverse cross section of the catheter of FIG. 7, taken along line 8-8. However, the shaft can alternatively be positioned eccentrically within the lumen of the tubular membrane 16, as for example in an embodiment (not shown) in which the shaft extends along and in contact with (e.g., secured to) the inner surface of the tubular membrane 16.

The agent delivery lumen 13 of the shaft 11 is in fluid communication with an agent delivery distal port 33 at the distal end of the agent delivery lumen 13. The agent delivery lumen 13 extends across the membrane 16 from the inner surface toward the outer surface of the membrane 16 (e.g., to agent delivery port 33 in a sidewall of the membrane), so that the agent delivery distal port 33 allows for flowing an agent from the lumen 13 into the agent containment pocket 32. In the illustrated embodiment, a short conduit 34, defining a distal end section of the agent delivery lumen 13, extends at a angle radially away from the shaft 11 between the agent delivery port 33 and the shaft 11. However, a variety of suitable configurations can be used, including one or more agent delivery ports on an outer surface of the shaft 11 in the embodiment in which the shaft is eccentrically positioned within the membrane 16 and secured to an inner surface thereof. Although the illustrated embodiment has a single agent delivery port 33, the shaft 11 can be provided with multiple agent delivery ports and/or agent delivery lumens in alternative embodiments (not shown). The catheter 10 can thus be configured for delivery of a single agent, or for sequential or simultaneous delivery of multiple agents.

As best illustrated in FIG. 9, showing a transverse cross section of FIG. 7, taken along line 9-9, the doughnut-shaped balloon 18 encircles and extends fully around circumference of the shaft 11 so that the balloon forms a 360° sealing surface against the wall of the body lumen 30, to fully isolate the agent containment pocket 32 from the blood flow within the body lumen 30.

In a method of delivering an agent to the patient's body lumen 30, the catheter 10 is introduced within the patient's body lumen 30. Preferably the catheter 10 is advanced within the lumen of a delivery catheter (not shown) and then positioned distal to the distal end of the delivery catheter (by distally advancing the catheter 10 or proximally withdrawing the delivery catheter), at a desired location in the body lumen 30. However, the catheter 10 can be introduced and advanced toward a desired treatment location in the body lumen 30 using a variety of suitable conventional methods, including slidably advancing the catheter 10 over a guidewire in an embodiment (not shown) having a guidewire lumen within shaft 11.

Once the catheter is at the desired location in the body lumen, each ring 17 is radially self-expanded. For example, sheath 20 is preferably proximally retracted while the catheter 10 is held stationary, although the catheter 10 can alternatively be distally advanced out the distal end of the sheath 20, to remove the radially restraining force of the sheath 20 from the rings 17. In a presently preferred embodiment, each ring 17 radially self-expands by pivoting away from the shaft, although the rings 17 can be configured such that the profile radially self-expands by a variety of suitable transformations including by unfolding, unwrapping, deforming, and the like. In the illustrated embodiment, the rings 17 transform from a thin oval shape in the collapsed configuration to a circular shape in the expanded configuration. The rings 17 are typically dimensioned such that the radially self-expanded rings are radially spaced away from the inner surface of the wall of the body lumen 30, to open the perfusion channel 21 and subsequently support the inflated balloons 18 while minimizing injury to the wall of the body lumen. However, the rings can alternatively be dimensioned to radially self-expand into contact with/snugly fitting the inner surface of the wall of the body lumen 30.

After the rings 17 are expanded, the balloons 18 are inflated by directing inflation fluid from the inflation lumen 12 into the interior chamber 19 of each balloon. The balloons 18 are inflated into contact with an inner surface of the patient's body lumen wall, with each inflated balloon 18 preferably extending fully around the inner circumference of the body lumen 30, as best illustrated in FIG. 9. The balloons 18 are typically configured to exert a sealing force against the body lumen wall, and may dilate/expand the body lumen wall, although the balloons are preferably configured to exert a radial force that does not cause harm or otherwise promote restenosis at the treatment location. In a presently preferred embodiment, the balloons 18 inflate at the same time, although the shaft 11 can alternatively be configured for sequentially inflating the balloons 18 independently of one another, by providing a separate inflation lumen for each balloon 18. Additionally, the inflation characteristics of the balloons 18 can be varied so that they do not inflate simultaneously into contact with the body lumen wall. For example, the distal balloon 18 can be formed to inflate after the proximal balloon at a higher inflation pressure than the proximal balloon, to facilitate flushing or otherwise minimizing blood from within the agent containment pocket 32 between the deployed sealing members.

With the sealing members 14, 15 thus deployed in the body lumen 30, an agent is delivered to the agent containment pocket 32. The agent flows from the agent delivery lumen 13, and out the port 33, and is held by the membrane 16 and sealing members 14, 15 in the agent containment pocket 32 so that the agent will absorb into or otherwise treat or act upon the wall of the body lumen 30. In one embodiment, the agent flow is started before at least the distal balloon 18 is fully inflated against the body lumen wall in order to flush the agent containment pocket 32 with agent (e.g., displace blood/body fluid with agent). In an alternative embodiment, the agent flow is started after the distal balloon is fully inflated to prevent or minimize the systemic release of the agent.

The membrane 16 is preferably a solid-walled, non-porous polymeric material to contain the agent within the agent containment pocket 32. A variety of suitable polymeric materials can be used to form the membrane 16 including polyurethanes, copolyamides such as polyether block amide (PEBAX) and styrenic block copolymers such as SYNPRENE. Although discussed primarily in terms of delivery of an agent through an agent delivery lumen of the shaft, the membrane 16 can additionally or alternatively be used for agent delivery, by impregnating or coating the membrane (e.g., the outer surface thereof) with an agent which will elute from the membrane while the device is deployed in the patient's body lumen.

The catheter shaft, or other components, preferably do not extend along or otherwise obstruct the outer surface of the membrane 16 between the balloons 18. As a result, the outer surface of the membrane 16 between the deployed sealing members 14, 15 extends directly adjacent to the inner surface of the body lumen wall, such that the containment pocket 32 extends fully around an inner circumference of the body lumen (see FIGS. 7 and 8). Additionally, the outer surface of the membrane 16 between the deployed sealing members 14, 15 is preferably radially spaced from the inner surface of the body lumen wall so that the agent containment pocket 32 is formed whether agent is flowing or not. The membrane is typically not supported by a self-expanding frame or other expanding structure between the proximal and distal sealing members, and thus is not radially forced against the inner surface of the wall of the body lumen 30.

A variety of suitable agents can be delivered using the catheter(s) and method(s) of the invention. The agents are typically intended for treatment and/or diagnosis of coronary, neurovascular, and/or other vascular disease, and may be useful as a primary treatment of the diseased vessel, or alternatively, as a secondary treatment in conjunction with other interventional therapies such as angioplasty or stent delivery. Suitable therapeutic agents include, but are not limited to, thrombolytic drugs, anti-inflammatory drugs, anti-proliferative drugs, drugs restoring and/or preserving endothelial function, and the like. A variety of bioactive agents can be used including but not limited to peptides, proteins, oligonucleotides, cells, and the like. A variety of diagnostic agents can be used according to the present invention. According to the present invention, agents described herein may be provided in a variety of suitable formulations and carriers including liposomes, polymerosomes, nanoparticles, microparticles, lipid/polymer micelles, and complexes of agents with lipid and/or polymers, and the like.

The dimensions of catheter 10 depend upon factors such as the catheter type and the size of the artery or other body lumen through which the catheter must pass. By way of example, the shaft 11 typically has an outer diameter of about 0.040 to about 0.075 inch (1.0 to 2.0 mm), and sheath 20 typically has an outer diameter of about 0.050 to about 0.085 inch (1.25 to 2.25 mm) and a wall thickness of about 0.0010 to about 0.0040 inch (0.04 to 0.10 mm). The membrane 16 length between the deployed sealing members 14, 15 is about 5.0 to about 50 mm, preferably about 10 to about 25 mm. Each inflated balloon 18 length is typically about 1 mm, and consequently, the length of the vessel wall which is contacted by the inflated balloons is significantly shorter than the length of the vessel wall which extends along the agent containment pocket/membrane. Typically, for coronary arteries, the proximal and distal balloons 18 inflate to a nominal outer diameter of about 1.0 to about 1.5 mm. The overall length of the catheter 10 may range from about 100 to about 150 cm, and is typically about 143 cm.

The shaft tubular members can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives. Although the shaft is illustrated as a dual lumen extrusion, a variety of suitable shaft configurations can be used including one or more of the tubular members formed of single or multiple layers or sections of tubing, as are conventionally known for catheter shaft design.

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

We claim:

1. A catheter for delivering an agent to an inner surface of a patient's body lumen wall, comprising:
a) an elongated shaft having an inflation lumen, and an agent delivery lumen which is in fluid communication with an agent delivery distal port;
b) a distal shaft section of the elongated shaft having a proximal sealing member and a distal sealing member secured thereto, wherein each sealing member reversibly expands from a low profile configuration to a deployed configuration, and comprises a ring which is biased to radially self-expand from a collapsed to a radially expanded configuration upon removal of a radially restraining force and a doughnut-shaped balloon which is on the self-expanding ring and which has an inflatable interior chamber in fluid communication with the inflation lumen; and
c) a tubular membrane extending from the proximal sealing member to the distal sealing member, having an outer surface, and an inner surface defining a perfusion channel extending through the tubular membrane with the rings in the expanded configuration, and which has the shaft extending in the perfusion channel, and the agent delivery lumen extends across the membrane from the inner towards the outer surface thereof, so that the catheter is configured to deliver an agent to an agent containment pocket which extends along the inner surface of the patient's body lumen wall between the deployed sealing members and which is formed by the outer surface of the membrane and the deployed sealing members.

2. The catheter of claim 1 wherein the doughnut-shaped balloons encircle and extend fully around a circumference of the shaft.

3. The catheter of claim 1 wherein the self-expanding ring is contained within the inflatable interior chamber of the balloon.

4. The catheter of claim 3 wherein the self-expanding ring is not bonded to the balloon.

5. The catheter of claim 3 including a tether member extending between the self-expanding ring and the catheter shaft which secures the ring to the shaft.

6. The catheter of claim 5 wherein the self-expanding ring and tether member are an integral, one-piece wire having a first section extending in the inflation lumen to form the tether member and a second, loop section forming the self-expanding ring.

7. The catheter of claim 1 wherein the membrane is bonded to an outer surface of each of the proximal and the distal balloons.

8. The catheter of claim 1 wherein the membrane is not supported by a self-expanding frame between the balloons, so that the membrane is connected to the sealing members such that radially self-expanding the rings is configured to open the perfusion channel through the membrane, and inflating the balloons is configured to position the outer surface of the portion of the membrane located between the deployed sealing members radially spaced from the inner surface of the patient's body lumen wall.

9. The catheter of claim 1 wherein the outer surface of the membrane is configured for positioning directly adjacent to the inner surface of the patient's body lumen, such that the containment pocket extends fully around an inner circumference of the body lumen.

10. The catheter of claim 1 wherein the catheter includes a sheath member slidably disposed on the shaft, which extends over and thereby collapses the self-expanding rings in an advanced configuration, and which has a retracted configuration with a distal end of the sheath proximal to each ring.

11. A method of performing a medical procedure, comprising
a) introducing within a patient's body lumen a catheter comprising
i) an elongated shaft having an inflation lumen and an agent delivery lumen,
ii) a distal shaft section of the elongated shaft having a proximal sealing member and a distal sealing member secured thereto, each sealing member reversibly expands from a low profile configuration to a deployed configuration and comprises a ring which is biased to radially self-expand from a collapsed to a radially expanded configuration upon removal of a radially restraining force, and a doughnut-shaped balloon on the self-expanding ring with an inflatable interior chamber in fluid communication with the inflation lumen, and
iii) a tubular membrane extending from the proximal sealing member to the distal sealing member, having an outer surface, and an inner surface defining a perfusion channel extending through the membrane with the rings in the expanded configuration, and which has the shaft extending in the perfusion channel, and the agent delivery lumen extending across the membrane from the inner towards the outer surface thereof;
b) radially self-expanding each ring by removing the radially restraining force therefrom;
c) inflating each balloon into contact with an inner surface of the patient's body lumen, each inflated balloon extending fully around an inner circumference of a wall of the body lumen; and
d) delivering an agent to an agent containment pocket which extends along the inner surface of the patient's body lumen wall between the deployed sealing members and which is formed by the outer surface of the membrane and the deployed sealing members.

12. The method of claim 11 wherein the membrane is not supported by a self-expanding frame between the balloons, so that radially self-expanding the rings in b) opens the perfusion channel through the membrane, and inflating the balloons positions the outer surface of the portion of the membrane located between the deployed sealing members radially spaced from the inner surface of the patient's body lumen wall.

13. The method of claim 12 wherein the outer surface of the membrane between the deployed sealing members extends directly adjacent to the inner surface of the patient's body lumen wall, such that the containment pocket extends fully around an inner circumference of the body lumen.

14. The method of claim 11 wherein the ring radially-self expands by pivoting away from the shaft, and radially collapses by pivoting down towards the shaft.

15. The method of claim 11 wherein the catheter includes a sheath member slidably disposed on the shaft, which extends over and thereby collapses the self-expanding rings in an advanced configuration, and which has a retracted configuration with a distal end of the sheath proximal to each ring, and wherein radially self-expanding each ring in b) comprises longitudinally displacing the sheath relative to the rings to position the sheath member in the retracted configuration.

16. A method of performing a medical procedure, comprising
a) introducing within a patient's body lumen a catheter comprising
i) an elongated shaft having an inflation lumen and an agent delivery lumen,
ii) a distal shaft section of the elongated shaft having a proximal sealing member and a distal sealing member secured thereto, each sealing member having a radially self-expanding component and an inflatable component, and
iii) a tubular membrane extending from the proximal sealing member to the distal sealing member, having an outer surface, and an inner surface defining a perfusion channel extending therethrough, and which has the shaft extending in the perfusion channel, and the agent delivery lumen extending across the membrane from the inner towards the outer surface thereof;
b) radially self-expanding each sealing member by removing the radially restraining force therefrom, and thereby radially expanding the tubular membrane to open the perfusion channel therethrough;
c) inflating each sealing member into contact with and extending fully around an inner circumference of an inner surface of a wall of the patient's body lumen, and thereby exerting a sealing force against the body lumen wall; and
d) delivering an agent to an agent containment pocket which extends along the inner surface of the patient's body lumen between the expanded sealing members and which is formed by the outer surface of the membrane and the expanded sealing members.
